(19) United States
(12) Patent Application Publication
(10) Pub. No.: US 2019/0159705 A1
(43) Pub. Date: May 30, 2019

(54) NON-INVASIVE GLUCOSE PREDICTION SYSTEM, GLUCOSE PREDICTION METHOD, AND GLUCOSE SENSOR

(71) Applicant: ELECTRONICS AND TELECOMMUNICATIONS RESEARCH INSTITUTE, Daejeon (KR)

(72) Inventors: Joo Yong SIM, Daejeon (KR); Chang-Geun AHN, Sejong (KR); Bong Kyu KIM, Daejeon (KR); Eun-Ju JEONG, Daejeon (KR); Kwang Hyo CHUNG, Daejeon (KR); Chul HUH, Daejeon (KR)

(21) Appl. No.: 16/204,950

(22) Filed: Nov. 29, 2018

(30) Foreign Application Priority Data
Nov. 29, 2017 (KR) 10-2017-0162169
Nov. 29, 2018 (KR) 10-2018-0150953

(51) Int. Cl. A61B 5/145; A61B 5/00

(52) U.S. Cl. CPC A61B 5/14532; A61B 5/0095; A61B 2576/00; A61B 5/7267; A61B 5/7275; A61B 5/6826

(57) ABSTRACT

A blood glucose prediction method comprising: acquiring a PAS signal by irradiating light to skin of the body, obtaining a photoacoustic image of the skin from the PAS signal, selecting at least one measurement location based on the photoacoustic image; and predicting the blood glucose based on a photoacoustic spectrum of a PAS signal corresponding to the at least one measurement location among the PAS signals, a blood glucose sensor, and a blood glucose prediction system are provided.

### CROSS-REFERENCE TO RELATED APPLICATION

This application claims priority to and the benefit of Korean Patent Application Nos. 10-2017-0162169 and 10-2018-0150953 filed in the Korean Intellectual Property Office on Nov. 29, 2017, and Nov. 29, 2018, respectively, the entire contents of which are incorporated herein by reference.

### BACKGROUND OF THE INVENTION

### (a) Field of the Invention

The present invention relates to a blood glucose prediction method, and a blood glucose sensor, and a blood glucose prediction system by non-invasive scheme.

### (b) Description of the Related Art

Diabetes is a disease of one in eleven people in the world. The diabetes is a significant economic burden for individuals, families, health systems, and the nation. If blood glucose (blood sugar) levels of diabetic patients is not maintained, diabetic patients will have serious complications, such as cardiovascular disease, kidney disease, and diabetic foot, which can cause serious discomfort in patients' life. Therefore, the blood glucose level should be regularly monitored and high blood glucose levels need to be addressed promptly.

In general, the blood glucose levels are determined from blood samples obtained invasively using an enzyme-containing electrochemical reaction sensor. Obtaining the blood through the process of stabbing a finger with a finger at this time may be a great inconvenience to a diabetic patient who needs to measure blood sugar several times a day, and the risk of infection is great. As non-invasive blood glucose measurement methods, a Raman spectroscopy, a diffuse reflection spectroscopy, a thermal emission spectroscopy, a near-infrared absorption spectroscopy, an mm-wave terahertz spectroscopy, a transdermal impedance spectroscopy, a sonophoresis, and an iontophoresis techniques have been developed. However, such a measurement method has a problem in that the precision is deteriorated depending on the secretion of skin tissue or the skin condition.

### SUMMARY OF THE INVENTION

An exemplary embodiment provides a method for predicting blood glucose in a body using a photoacoustic spectrography.

Another exemplary embodiment provides a sensor for predicting blood glucose in a body using a photoacoustic spectrography.

Yet another exemplary embodiment provides a system for predicting blood glucose in a body using a photoacoustic spectrography.

According to an exemplary embodiment, a method for predicting blood glucose in a body using a photoacoustic spectrography (PAS) is provided. The method includes: acquiring a PAS signal by irradiating light to skin of the body; obtaining a photoacoustic image of the skin from the PAS signal; selecting at least one measurement location based on the photoacoustic image; and predicting the blood glucose based on a photoacoustic spectrum of a PAS signal corresponding to the at least one measurement location among the PAS signals.

The acquiring a PAS signal by irradiating light to skin of the body may include irradiating the light of a plurality of wavelengths in a predetermined band into a predetermined area of the skin.

The irradiating the light of a plurality of wavelengths in a predetermined band into a predetermined area of the skin may include irradiating the light into the predetermined area while gradually increasing a size of the wavelength of the light within a near-infrared (NIR) band or a mid-infrared (MIR) band.

The irradiating the light of a plurality of wavelengths in a predetermined band into a predetermined area of the skin may include irradiating the light into the predetermined area while gradually decreasing a size of the wavelength of the light within a near-infrared (NIR) band or a mid-infrared (MIR) band.

The irradiating the light of a plurality of wavelengths in a predetermined band into a predetermined area of the skin may include irradiating the light into the predetermined area in a zigzag direction, concentrically, or spirally.

The selecting at least one measurement location based on the photoacoustic image may include selecting a location with a relatively low brightness in the photoacoustic image as the at least one measurement location.

The location with a relatively low brightness in the photoacoustic image may indicate a location which does not include a skin hole connected to a gland of the skin.

The location with a relatively low brightness in the photoacoustic image may indicate a valley of a fingerprint when the skin is a finger skin.

The selecting at least one measurement location based on the photoacoustic image may include selecting a location at which a change in photoacoustic spectrum is relatively small during a predetermined time interval as the at least one measurement location.

The predicting the blood glucose based on a photoacoustic spectrum of a PAS signal corresponding to the at least one measurement location among the PAS signals may include: transmitting information about the photoacoustic spectrum to a computing processor or a server; and receiving information on blood glucose predicted based on machine learning using the photoacoustic spectrum from the computing processor or the server.

According to another exemplary embodiment, a sensor for predicting blood glucose in a body using a photoacoustic spectrography (PAS) is provided. The sensor includes: a light emitter configured to emit light to skin of the body; an acoustic resonator configured to amplifying a PAS signal using at least one cavity, wherein the PAS signal is generated by the skin after absorbing heat of the light; and a photoacoustic detector configured to acquire the PAS signal amplified by the acoustic resonator.

The light emitter may be configured to emit the light into a predetermined area of the skin while gradually increasing or decreasing a size of a wavelength of the light within a near-infrared (NIR) band or a mid-infrared (MIR) band.

The light emitter may further be configured to emit the light into the predetermined area in a zigzag direction, concentrically, or spirally.

The acoustic resonator may include a first cavity and a second cavity, the light is emitted onto the skin through the first cavity, and the PAS signal generated from the skin may be detected by the photoacoustic detector connected to an end of the second cavity.

The photoacoustic detector may include a microphone and an amplifier, and a resonance frequency of the microphone may correspond with a resonance frequency of the acoustic resonator within an error range.

The sensor may further include a photoacoustic analyzer and a communication unit, wherein the photoacoustic analyzer may be configured to transmit information about the PAS signal to a computation device or a server through the communication unit and receive information about the blood glucose predicted based on machine learning using the photoacoustic spectrum of the PAS signal from the computation device or the server through the communication unit.

According to yet another exemplary embodiment, a system for predicting blood glucose in a body using a photoacoustic spectrography (PAS) is provided. The system includes; a blood glucose sensor configured to amplify a PAS signal generated from skin of the body by irradiating light to the skin and acquire the PAS signal; and a photoacoustic analyzer configured to obtain a photoacoustic image of the skin from the PAS signal, selects at least one measurement location based on the photoacoustic image, and predict the blood glucose based on a photoacoustic spectrum of a PAS signal corresponding to the at least one measurement location.

The photoacoustic analyzer may be configured to transmit information about a photoacoustic spectrum of a PAS signal corresponding to the at least one measurement location to a computation device or a server via a wired and/or wireless network, and receive information on the blood glucose which is predicted based on machine learning using the photoacoustic spectrum from the computation device or the server.

The photoacoustic analyzer may be configured to select a location with a relatively low brightness in the photoacoustic image as the at least one measurement location.

The photoacoustic analyzer may be configured to select, as the at least one measurement location, a location in the photoacoustic image where change in the photoacoustic spectrum is relatively small for a predetermined time duration.

### BRIEF DESCRIPTION OF THE DRAWINGS

FIG. 1 is a block diagram illustrating a blood glucose sensor according to an exemplary embodiment.

FIG. 2 is graphs illustrating an operation of the blood glucose sensor and determination of a diameter of a light beam and a frequency of light beam according to an exemplary embodiment.

FIG. 3 is a conceptual diagram illustrating a relative positional relationship between a blood glucose sensor and a body according to an exemplary embodiment.

FIG. 4 is a photoacoustic image of a PAS signal corresponding to each skin location according to an exemplary embodiment.

FIG. 5 is a graph illustrating photoacoustic spectrum of each skin location according to an exemplary embodiment.

FIG. 6A is a schematic diagram illustrating a change of a photoacoustic spectrum of the PAS signal in time according to an exemplary embodiment.

FIG. 6B is a diagram illustrating photoacoustic spectrum and a change of the photoacoustic spectrum at the location shown in FIG. 6A.

FIG. 7 is a flowchart illustrating a method of predicting blood glucose levels by a blood glucose prediction system according to an exemplary embodiment of the present invention.

FIG. 8 is a schematic diagram illustrating a structure of a non-invasive type blood glucose sensor according to an exemplary embodiment.

FIG. 9 is a graph for determining the optimal dimensions of the acoustic resonator according to an exemplary embodiment.

FIG. 10 is a block diagram illustrating a photoacoustic analyzer of the blood glucose prediction system according to another embodiment.

### DETAILED DESCRIPTION OF THE EMBODIMENTS

Hereinafter, exemplary embodiments of the present disclosure will be described in detail with reference to the accompanying drawings so that those skilled in the art may easily practice the present disclosure. However, the present disclosure may be modified in various different ways and is not limited to embodiments described herein. In the accompanying drawings, portions unrelated to the description will be omitted in order to obviously describe the present disclosure, and similar reference numerals will be used to describe similar portions throughout the present specification.

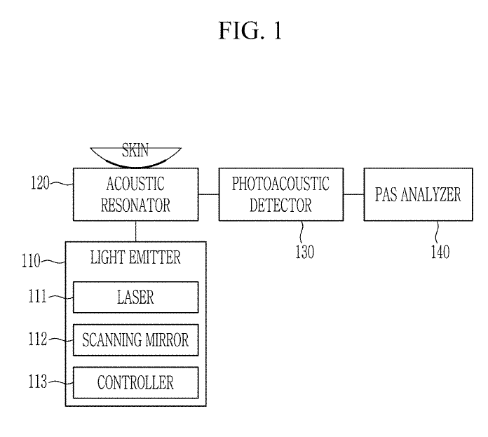

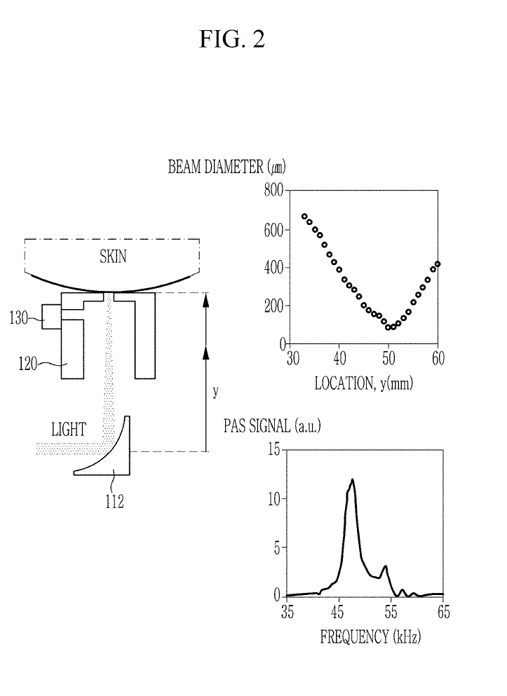

FIG. 1 is a block diagram illustrating a blood glucose sensor according to an exemplary embodiment, and FIG. 2 is graphs illustrating an operation of the blood glucose sensor and determination of a diameter of a light beam and a frequency of light beam according to an exemplary embodiment.

Referring to FIG. 1, a blood glucose sensor 100 according to an exemplary embodiment includes a light emitter 110, an acoustic resonator 120, and a photoacoustic detector 130.

The light emitter 110 includes a laser 111, a scanning mirror 112, and a controller unit 113. The controller 113 may control the scanning mirror 112 so that a short wavelength light output from the laser 111 scans the skin. For example, the light output from the laser 111 scans a predetermined area on the skin by the scanning mirror 112 under the control of the controller 113. That is, the light emitter 110 may irradiate the light onto a predetermined area of the skin in a zig-zag direction, along with a concentric circle, or in a spiral shape. The light emitter 110 may emit the light having various wavelengths in a wavelength band at which the blood glucose may be most effectively measured. For example, the light emitter 110 may irradiate the light of a plurality of wavelengths into a predetermined band while gradually increasing or decreasing a size of the wavelength of the light within a near-infrared (NIR) band or a mid-infrared (MIR) band so that a photoacoustic spectrum indicating a change in the magnitude of the PAS signal for each wavelength can be obtained. The wavelength band of light emitted from the light emitter 110 may vary depending on the kind of substance in the body to be measured.

The acoustic resonator 120 may amplify a photoacoustic spectrography (PAS) signal generated after the laser output from the light emitter 110 is irradiated onto the skin through at least one cavity included in the acoustic resonator 120. The PAS signal is an acoustic signal generated when the light such as a laser is radiated onto a narrow area of the skin surface at a high density and then the skin tissue absorbs heat of the light to expand instantaneously. At this time, the PAS signal is emitted in a pulse shape within a very short time interval (nanosecond scale), and the size of the PAS signal is amplified due to the structural characteristic of the resonance structure 120 and is detected by the photoacoustic detector 130.

Referring to FIG. 2, when the light steered by the scanning mirror 112 is irradiated onto the skin, the PAS signal is amplified by the acoustic resonator 120 and then is measured by the photoacoustic detector 130. The two graphs on the right side of FIG. 2 are graphs showing the relationship between the location of the scanning mirror 112 for the sample and the beam diameter and the frequency response characteristics of the resonator structure 120, respectively. Referring to FIG. 2, when the location y from the scanning mirror 112 to the skin is 50 mm, the diameter of the beam portion (focus) where light comes into contact with the skin is about 90 μm (i.e., minimum). This is an experimentally obtained value and may vary depending on the specification of the scanning mirror 112 and so on. That is, the location from the scanning mirror 112 to the skin may be determined so as to minimize the beam diameter of the light. Also, the resonance frequency of the acoustic resonator 120 is about 47.5 kHz, so that the light emitter 110 according to the exemplary embodiment may irradiate the light at intervals of 45 kHz to 50 kHz.

The photoacoustic detector 130 includes a photoacoustic receiver 131 (for example, a microphone), and further includes a filter 132 and an amplifier 133 for signal processing of the PAS signal. Alternatively, the photoacoustic detector 130 may amplify the PAS signal through a lock-in amplifier (not shown).

The blood glucose sensor 100 according to an exemplary embodiment may further include a photoacoustic analyzer (PAS analyzer) 140 and a communication unit (not shown).

The photoacoustic analyzer 140 may analyze the PAS signal to predict the blood glucose level in the body. The photoacoustic analyzer 140 may directly predict the blood glucose level by analyzing the PAS signal. Alternatively, the photoacoustic analyzer 140 transmits information about the PAS signal to an external computation device or a server when the amount of calculation required for predicting the blood glucose is excessive and receives information about the blood glucose predicted based on machine learning using the PAS signal by the external computation device or the server. That is, the photoacoustic analyzer 140 may transmit the information about the PAS signal to the external computation device or the server through the communication unit, and receive the blood glucose level determined based on the machine learning by using the photoacoustic spectrum of the PAS signal from the computation device or the server through the communication unit. The external computation device may be a mobile communication device connected to a short-range wireless communication network (e.g., WLAN, Bluetooth), or a personal computer of a user connected to a wired network (e.g., LAN, USB interface), or a remote server connected to a long-distance wireless communication network (e.g., WAN). The functions of the photoacoustic analyzer 140 described below include photoacoustic analyzing functions used by the external computation device or the server.

The photoacoustic analyzer 140 according to another exemplary embodiment may obtain a photoacoustic image corresponding to each wavelength from the PAS signal generated from the light having various wavelengths, and may predict blood glucose levels in the body by performing a machine learning based on a plurality of photoacoustic images corresponding to the respective wavelengths. That is, the photoacoustic analyzer 140 (or an external computation device or a server) may perform deep learning using a plurality of photoacoustic images corresponding to the respective wavelengths. The photoacoustic analyzer 140 may apply regression analysis using a convolutional neural network (CNN) for the deep learning.

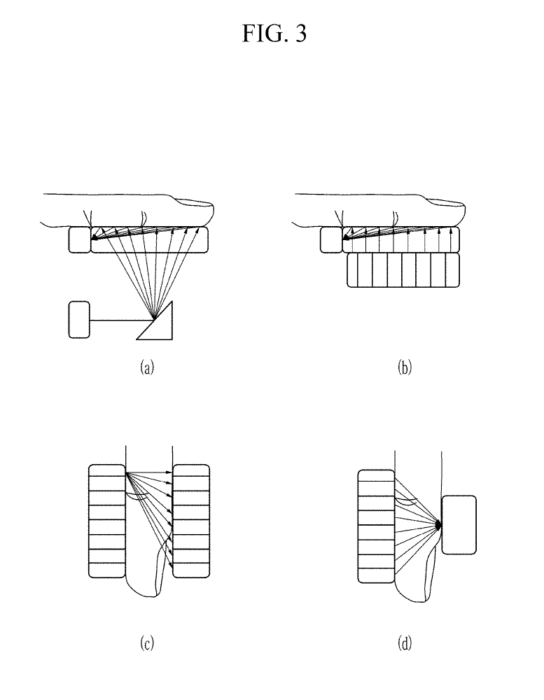

FIG. 3 is a conceptual diagram illustrating a relative positional relationship between a blood glucose sensor and a body according to an exemplary embodiment.

Referring to FIG. 3, the light emitter 110, the acoustic resonator 120, and the photoacoustic detector 130 are located on one side of the skin. Referring to (c) and (d) of FIG. 3, the light emitter 110 is located on a different side from the acoustic resonator 120. For example, when the blood glucose sensor 100 according to the exemplary embodiment is attached to a wrist, a finger, or the like, the blood glucose sensor 100 shown in (a) and (b) of FIG. 3 performs the light emission and the photoacoustic detection at one side of the wrist, the finger, etc. In contrast, the blood glucose sensor 100 as shown in (c) and (d) generates the light at one side of the wrist or the finger, and light passing through the body reaches the acoustic resonator 120 at the opposite side of the finger or the wrist.

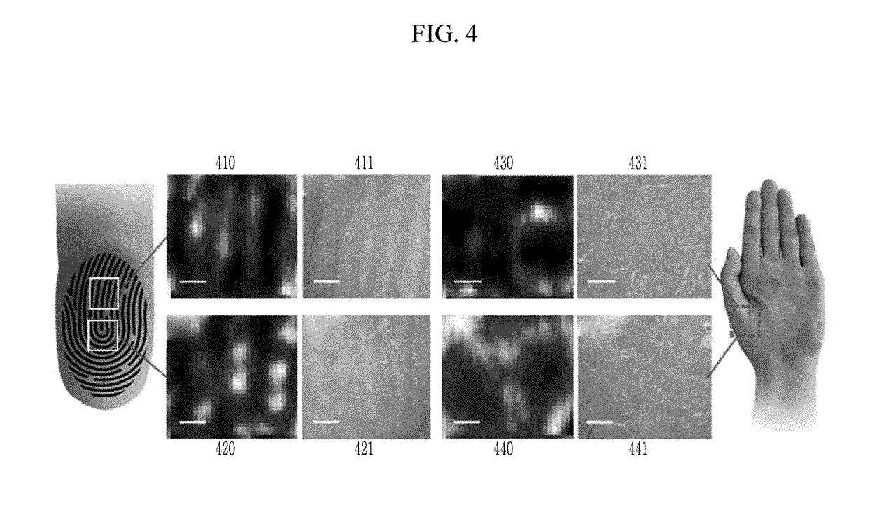

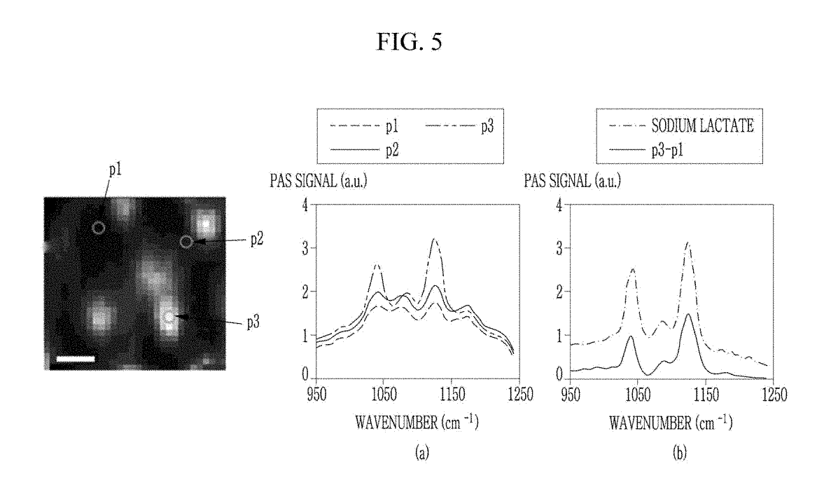

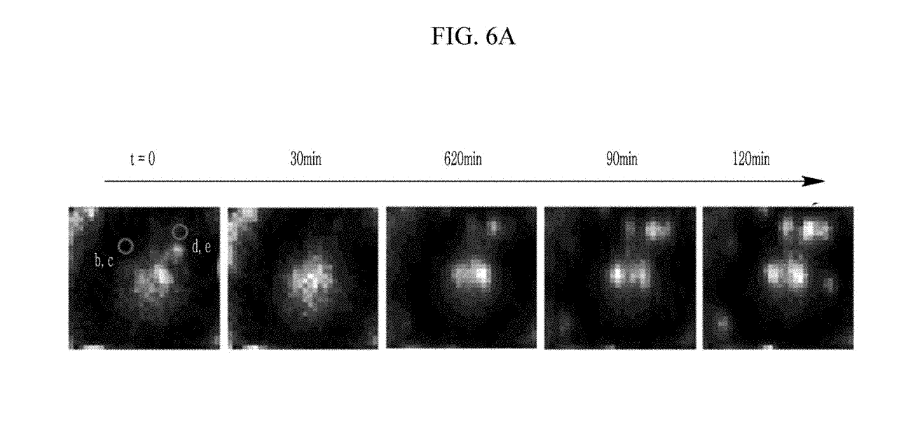

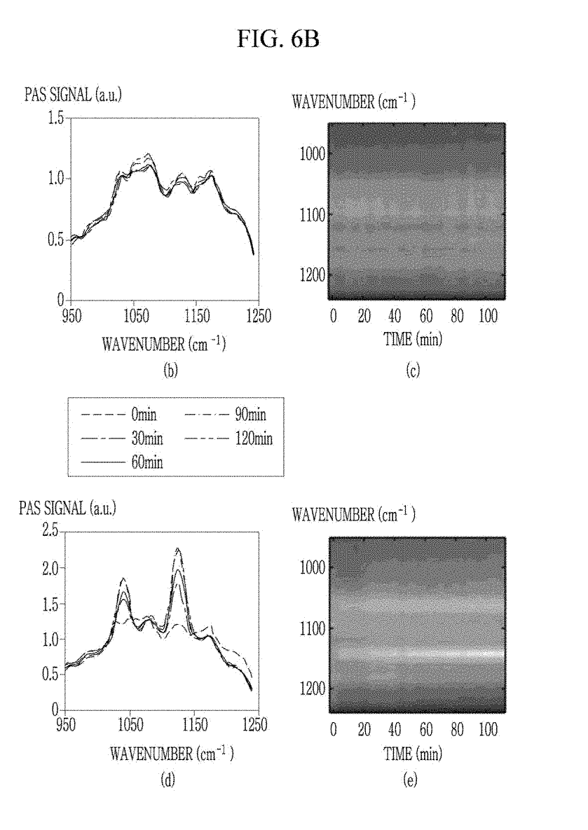

FIG. 4 is a photoacoustic image of a PAS signal corresponding to each skin location according to an exemplary embodiment, FIG. 5 is a graph illustrating photoacoustic spectrum of each skin location according to an exemplary embodiment, FIG. 6A is a schematic diagram illustrating a change of a photoacoustic spectrum of the PAS signal in time according to an exemplary embodiment, and FIG. 6B is a diagram illustrating photoacoustic spectrum and a change of the photoacoustic spectrum at the location shown in FIG. 6A.

The photoacoustic analyzer 140 according to an exemplary embodiment may generate a two-dimensional photoacoustic image of the skin and select at least one measurement location to be used in the blood glucose prediction in the two-dimensional photoacoustic image. FIG. 4, photoacoustic images 410 and 420 generated from the PAS signal acquired at two positions of the finger and photoacoustic images 430 and 440 generated from the PAS signal acquired at two positions of the palm of the hand are shown. According to the exemplary embodiment, a photoacoustic image may be generated based on a PAS signal acquired by irradiating the light on a finger or a part of the palm from the blood glucose sensor 100, and the photoacoustic analyzer 140 may select at least one measurement location to be used for predicting blood glucose. The at least one measurement location for use in predicting blood glucose in the photoacoustic image is selected to eliminate the effect of secretions that can degrade the accuracy of blood glucose measurements such as sweat, sebum, etc., which are secreted from the skin pores associated with the glands of the skin. That is, the photoacoustic analyzer 140 according to the exemplary embodiment may analyze the photoacoustic image and determine a location where the secretion is relatively less discharged as the measurement location for predicting the blood glucose level.

Referring to FIG. 5, the photoacoustic spectrum of the PAS signal acquired at the points p1, p2, and p3 are shown in a graph (a). The photoacoustic spectrum corresponding to p3, which is the brightest point in the photoacoustic image, differs from the photoacoustic spectrum at other points. From the graph (b), it can be seen that the photoacoustic spectrum of p3 is similar to that of sodium lactate, which is obtained by subtracting the photoacoustic spectrum of p1 from the photoacoustic spectrum of p3. In other words, it may be learned that bright areas such as p3 in the photoacoustic image are affected by the secretions of the body such as sodium lactate. Therefore, a dark area in the photoacoustic image, which is expected to have the least effect by the secretions, such as p1, may be determined as the measurement location for determining the blood glucose level.

Also, skin conditions over time may be considered to select the measurement location. Referring to FIGS. 6A and 6B, graphs (b) and (c) show the photoacoustic spectrum acquired at the first position in the photoacoustic image and time-dependent changes of the photoacoustic spectrum, respectively. The graphs (d) and (e) show the photoacoustic spectrum acquired at the second position different from the first position in the photoacoustic image and time-dependent changes of the photoacoustic spectrum, respectively. Referring to FIG. 6A, the brightness of the second position becomes brighter as time elapses, and the results are shown in graphs (d) and (e) of FIG. 6B, respectively.

As described above, by using the photoacoustic image generated based on the PAS signal, the influence of the secretions of the skin, the state of the skin, the uniformity of the skin surface, and the like may be effectively removed from the measurement result and the accuracy of the blood glucose prediction may be enhanced. Further, by using the photoacoustic spectrum of the measurement location corresponding to the part of the photoacoustic images, the computing resources consumed in the computation device for performing the machine learning can be reduced, and the speed of the machine learning for the blood glucose prediction may be remarkably improved.

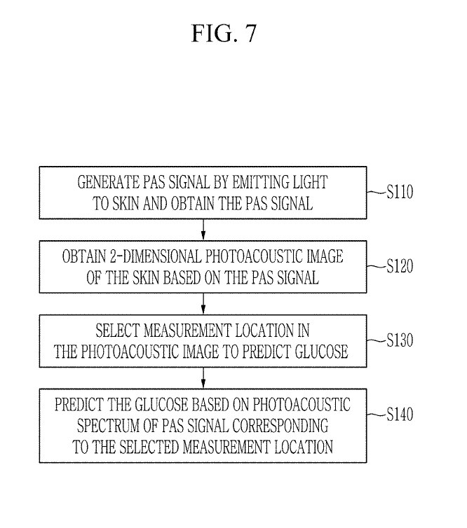

FIG. 7 is a flowchart illustrating a method of predicting blood glucose levels by a blood glucose prediction system according to an exemplary embodiment of the present invention.

Referring to FIG. 7, the light emitter 110 of the blood glucose sensor 100 irradiates light to the skin to generate a PAS signal, and a PAS signal is acquired by the photoacoustic detector 130 (S110). The laser 111 of the light emitter 110 may emit a plurality of lasers having wavelengths of various sizes and controls the scanning mirror 112 so that the light having the plurality of wavelengths is irradiated within a predetermined area of the skin. In addition, the PAS signal generated from the skin may be amplified by the acoustic resonator 120.

Then, the photoacoustic analyzer 140 (or an external computation device or a server receiving the information about the PAS signal from the photoacoustic analyzer 140) obtains a two-dimensional photoacoustic image of the skin from the PAS signal (S120), and selects at least one measurement location to be used for predicting blood glucose based on the photoacoustic image (S130). According to the exemplary embodiment, the at least one measurement location may be selected in order of low lightness in the photoacoustic image. For example, when n measurement locations for predicting blood glucose are required, the photoacoustic analyzer 140 may select the n measurement locations in order of low lightness in the photoacoustic image. The area of the photoacoustic image that has a relatively low lightness may indicate an area which does not include a skin hole connected to the gland of the skin. Alternatively, the area of the photoacoustic image where the brightness is relatively low may indicate a valley of a fingerprint when the skin is the finger skin. Also, the photoacoustic analyzer 140 may select an area of the photoacoustic image having a relatively small change in the photoacoustic spectrum as a measurement location for predetermined time duration.

Thereafter, the photoacoustic analyzer 140 predicts the blood glucose based on the photoacoustic spectrum of the PAS signal corresponding to the selected measurement location (S140). To acquire the photoacoustic spectrum of the PAS signal corresponding to the selected measurement location, the photoacoustic analyzer 140 may perform a wavenumber scan at the selected measurement location. The photoacoustic analyzer 140 may perform the machine learning using the photoacoustic spectrum of the PAS signal corresponding to the selected measurement location (or receive a result of the machine learning performed by the computation device or the server which are located outside therefor) and may predict the blood glucose according to the result of the machine learning. At this time, the photoacoustic analyzer 140 may transmit the information about the selected measurement location in the photoacoustic image and the photoacoustic image to the external computing device or the server through the communication unit to reduce the calculation load (alternatively, transmit the photoacoustic spectrum), and use the results of the machine learning performed by the external computing device or the server.

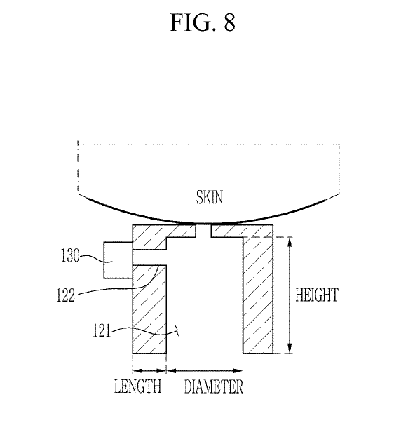

FIG. 8 is a schematic diagram illustrating a structure of a non-invasive type blood glucose sensor according to an exemplary embodiment.

Referring to FIG. 8, the acoustic resonator 120 according to an exemplary embodiment includes a main cavity 121 and a branch cavity 122. The light emitted from the light emitter 110 passes through the main cavity 121 and is irradiated to the skin. The PAS signal generated from the skin may be amplified according to the resonant characteristics of the acoustic resonator 120 and may be detected by the photoacoustic detector 130 located at the end of the branch cavity 122. The resonant characteristics of the acoustic resonator 120 according to the exemplary embodiment may vary depending on the material of the acoustic resonator 120, the height of the main cavity 121, the diameter of the main cavity 121, and the length of the branch cavity 122. The resonance frequency of the acoustic resonator 120 corresponds with the resonance frequency of the microphone included in the photoacoustic detector 130 within an error range.

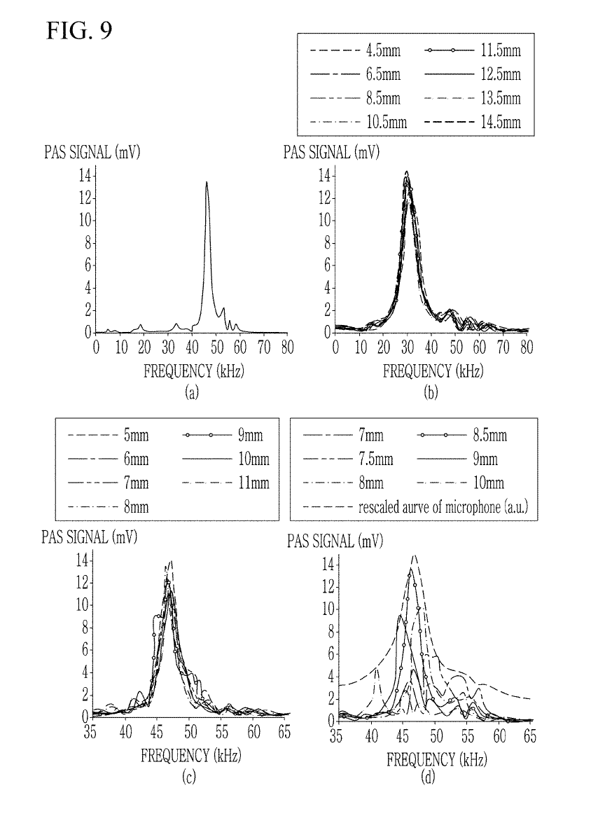

FIG. 9 is a graph for determining the optimal dimensions of the acoustic resonator according to an exemplary embodiment.

FIG. 9 is a graph showing frequency characteristics of the optimized acoustic resonator 120. Graph (b) is a graph showing frequency characteristics according to height variation of the main cavity 121. Graph (c) is a graph showing frequency characteristics according length variation of the branch cavity 122. Graph (d) is a graph showing frequency characteristics according to diameter variation of the main cavity 121. As shown in the graph (b), the height of the main cavity 121 varies from 4.5 mm to 14.5 mm, and it is confirmed that the higher the height of the main cavity 121, the better the resonance occurred. In the graph (c), the length of the branch cavity 122 is changed from 5 mm to 11 mm, and it is confirmed that the shorter the length of the branch cavity 122, the better the resonance occurred. In the graph (d), the diameter of the main cavity 121 is changed from 7 mm to 10 mm. In this case, different frequency characteristics are exhibited, and it is confirmed that the resonance occurred best when the diameter is 8.5 mm. The dashed line in the graph (d) is the rescaled frequency response curve of the photoacoustic receiver 131, such as a microphone.

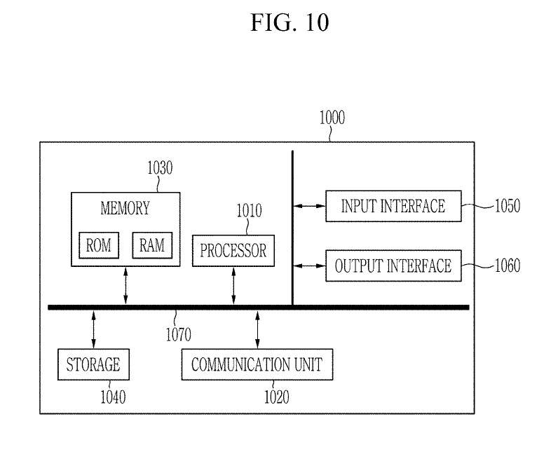

FIG. 10 is a block diagram illustrating a photoacoustic analyzer of the blood glucose prediction system according to another embodiment.

The blood glucose prediction system according to an exemplary embodiment may be implemented as a computer system, for example a computer readable medium. Referring to FIG. 10, a computer system 1000 may include at least one of processor 1010, a memory 1030, an input interface 1050, an output interface 1060, and storage 1040. The computer system 1000 may also include a communication device 1020 coupled to a network. The processor 1010 may be a central processing unit (CPU) or a semiconductor device that executes instructions stored in the memory 1030 or storage 1040. The memory 1030 and the storage 1040 may include various forms of volatile or non-volatile storage media. For example, the memory may include read only memory (ROM) or random access memory (RAM). In the exemplary embodiment of the present disclosure, the memory may be located inside or outside the processor, and the memory may be coupled to the processor through various means already known.

Thus, embodiments of the present invention may be embodied as a computer-implemented method or as a non-volatile computer-readable medium having computer-executable instructions stored thereon. In the exemplary embodiment, when executed by a processor, the computer-readable instructions may perform the method according to at least one aspect of the present disclosure. The communication device 1020 may transmit or receive a wired signal or a wireless signal.

On the contrary, the embodiments of the present invention are not implemented only by the apparatuses and/or methods described so far, but may be implemented through a program realizing the function corresponding to the configuration of the embodiment of the present disclosure or a recording medium on which the program is recorded. Such an embodiment can be easily implemented by those skilled in the art from the description of the embodiments described above. Specifically, methods (e.g., network management methods, data transmission methods, transmission schedule generation methods, etc.) according to embodiments of the present disclosure may be implemented in the form of program instructions that may be executed through various computer means, and be recorded in the computer-readable medium. The computer-readable medium may include program instructions, data files, data structures, and the like, alone or in combination. The program instructions to be recorded on the computer-readable medium may be those specially designed or constructed for the embodiments of the present disclosure or may be known and available to those of ordinary skill in the computer software arts. The computer-readable recording medium may include a hardware device configured to store and execute program instructions. For example, the computer-readable recording medium can be any type of storage media such as magnetic media like hard disks, floppy disks, and magnetic tapes, optical media like CD-ROMs, DVDs, magneto-optical media like floptical disks, and ROM, RAM, flash memory, and the like. Program instructions may include machine language code such as those produced by a compiler, as well as high-level language code that may be executed by a computer via an interpreter, or the like.

While this invention has been described in connection with what is presently considered to be practical example embodiments, it is to be understood that the invention is not limited to the disclosed embodiments, but, on the contrary, is intended to cover various modifications and equivalent arrangements included within the spirit and scope of the appended claims.

What is claimed is:

1. A method for predicting blood glucose in a body using a photoacoustic spectrography (PAS), comprising:
acquiring a PAS signal by irradiating light to skin of the body;
obtaining a photoacoustic image of the skin from the PAS signal;
selecting at least one measurement location based on the photoacoustic image; and
predicting the blood glucose based on a photoacoustic spectrum of a PAS signal corresponding to the at least one measurement location among the PAS signals.

2. The method of claim 1, wherein the acquiring a PAS signal by irradiating light to skin of the body comprises
irradiating the light of a plurality of wavelengths in a predetermined band into a predetermined area of the skin.

3. The method of claim 2, wherein the irradiating the light of a plurality of wavelengths in a predetermined band into a predetermined area of the skin comprises
irradiating the light into the predetermined area while gradually increasing a size of the wavelength of the light within a near-infrared (NIR) band or a mid-infrared (MIR) band.

4. The method of claim 2, wherein the irradiating the light of a plurality of wavelengths in a predetermined band into a predetermined area of the skin comprises
irradiating the light into the predetermined area while gradually decreasing a size of the wavelength of the light within a near-infrared (NIR) band or a mid-infrared (MIR) band.

5. The method of claim 2, wherein the irradiating the light of a plurality of wavelengths in a predetermined band into a predetermined area of the skin comprises
irradiating the light into the predetermined area in a zigzag direction, concentrically, or spirally.

6. The method of claim 1, wherein the selecting at least one measurement location based on the photoacoustic image comprises
selecting a location with a relatively low brightness in the photoacoustic image as the at least one measurement location.

7. The method of claim 6, wherein the location with a relatively low brightness in the photoacoustic image indicates a location which does not include a skin hole connected to a gland of the skin.

8. The method of claim 6, wherein the location with a relatively low brightness in the photoacoustic image indicates a valley of a fingerprint when the skin is a finger skin.

9. The method of claim 6, wherein the selecting at least one measurement location based on the photoacoustic image comprises
selecting a location at which a change in photoacoustic spectrum is relatively small during a predetermined time interval as the at least one measurement location.

10. The method of claim 1, wherein the predicting the blood glucose based on a photoacoustic spectrum of a PAS signal corresponding to the at least one measurement location among the PAS signals comprises:
transmitting information about the photoacoustic spectrum to a computing processor or a server; and
receiving information on blood glucose predicted based on machine learning using the photoacoustic spectrum from the computing processor or the server.

11. A sensor for predicting blood glucose in a body using a photoacoustic spectrography (PAS), comprising:
a light emitter configured to emit light to skin of the body;
an acoustic resonator configured to amplifying a PAS signal using at least one cavity, wherein the PAS signal is generated by the skin after absorbing heat of the light; and
a photoacoustic detector configured to acquire the PAS signal amplified by the acoustic resonator.

12. The sensor of claim 11, wherein the light emitter configured to emit the light into a predetermined area of the skin while gradually increasing or decreasing a size of a wavelength of the light within a near-infrared (NIR) band or a mid-infrared (MIR) band.

13. The sensor of claim 12, wherein the light emitter further configured to emit the light into the predetermined area in a zigzag direction, concentrically, or spirally.

14. The sensor of claim 11, wherein the acoustic resonator includes a first cavity and a second cavity, the light is emitted onto the skin through the first cavity, and the PAS signal generated from the skin is detected by the photoacoustic detector connected to an end of the second cavity.

15. The sensor of claim 11, wherein the photoacoustic detector includes a microphone and an amplifier, and a resonance frequency of the microphone corresponds with a resonance frequency of the acoustic resonator within an error range.

16. The sensor of claim 11, wherein the sensor further comprises a photoacoustic analyzer and a communication unit, wherein the photoacoustic analyzer is configured to transmit information about the PAS signal to a computation device or a server through the communication unit and receive information about the blood glucose predicted based on machine learning using the photoacoustic spectrum of the PAS signal from the computation device or the server through the communication unit.

17. A system for predicting blood glucose in a body using a photoacoustic spectrography (PAS),
a blood glucose sensor configured to acquire a plurality of PAS signals corresponding to a plurality of wavelengths by irradiating light having the plurality of wavelengths to the skin; and
a photoacoustic analyzer configured to obtain a plurality of photoacoustic images of skin of the body from the plurality of PAS signals, and predict the blood glucose through machine learning performed based on the plurality of photoacoustic images, wherein the plurality of photoacoustic images corresponds to the plurality of wavelengths of the light, respectively.

18. The system of claim 17, wherein the photoacoustic analyzer is configured to transmit the plurality of photoacoustic images to a computation device or a server which are located outside of the system via a wired and/or wireless network, and receive information on the blood glucose which is predicted based on machine learning performed based on the plurality of photoacoustic images from the computation device or the server.

19. The system of claim 17, wherein the photoacoustic analyzer is configured to predict the blood glucose by performing the machine learning through regression analysis using a convolutional neural network (CNN).

20. The system of claim 18, wherein the machine learning performed by the computation device or the server includes through regression analysis using a convolutional neural network (CNN).
